(19) United States
(12) Patent Application Publication
(10) Pub. No.: US 2026/0365984 A1
(43) Pub. Date: Nov. 19, 2026

(54) METHODS FOR GENERATING PREVASCULARIZED 3D CELL AGGREGATES

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); ETABLISSEMENT FRANÇAIS DU SANG (EFS), La Plaine Saint Denis (FR); UNIVERSITE COTE D'AZUR, Nice (FR); UNIVERSITÉ TOULOUSE III - PAUL SABATIER, Toulouse (FR)

(72) Inventors: Louis CASTEILLA, Toulouse (FR); Audrey CARRIERE, Toulouse (FR); Laurent MALAQUIN, Toulouse (FR); Christian DANI, Nice (FR); Laurence VAYSSE, Toulouse (FR); Mélanie ESCUDERO, Toulouse (FR); Gozde EKE, Vienne (AT)

(21) Appl. No.: 19/139,561

(22) PCT Filed: Dec. 19, 2023
(86) PCT No.: PCT/EP2023/086673

§ 371 Date: Jun. 16, 2025

(30) Foreign Application Priority Data
Dec. 20, 2022 (EP) 22306953.5
Feb. 2, 2023 (EP) 23305132.5

(51) Int. Cl. C12N 5/077; G01N 33/50

(52) U.S. Cl. CPC C12N 5/0653; G01N 33/5044; C12N 2501/15; C12N 2501/155; C12N 2501/33; C12N 2501/998; C12N 2506/1384; C12N 2506/28; C12N 2513/00

(57) ABSTRACT

When cultured in adequate 3D conditions, stem cells can undergo in-vivo like morphogenesis and turn into self-organized complex structures called organoids. Various avascular and vascular 3D models of tissue have already been developed. Regarding brown adipose tissue (BAT), main focus was given to the obtention of functional brown/beige adipocytes and not much consideration to other cell compartments. These tissue constructs still suffer from a lack of reproducibility and the failure of long-term culture maintenance. A solution to overcome these limitations is to provide a suitable microenvironment through engineering approaches. Accordingly, the present invention relates to the in vitro use of a TGF-β inhibitor for increasing the viability of a 3D cell aggregate and to an in vitro method for generating a prevascularized 3D cell aggregate comprising the step of contacting mesenchymal stroma/stem cells and endothelial cells with a differentiation medium and a TGF-β inhibitor.

### FIELD OF THE INVENTION

The present invention is in the field of medicine, in particular tissue engineering.

### BACKGROUND OF THE INVENTION

Three dimensions (3D) cell culture models have become a reference in the field of cell culture in order to reproduce the functions and architecture of tissues. Two dimensions (2D) models do not fully take into account the spatial organization of cells and their interactions with each other. Developing new 3D models makes it possible to obtain a better understanding of the development of pathologies associated with tissues but also to test the penetration of drugs through a tissue and the associated cellular response. A classic cell aggregation method involves centrifuging cells to be studied to generate 3D cell aggregates. However, this kind of method actually only makes it possible to obtain small 3D cell aggregates containing a limited number of cells. Even by increasing the number of centrifuged cells, it is not possible to date to obtain 3D cell aggregates of greater volume because the cells in the core of the aggregate become necrotic due to a restricted access to nutrients and to oxygen. The limited size of the 3D cell aggregates obtained does not allow the generation of satisfying tissue substitutes and the development of new culture systems is necessary to improve current therapeutic strategies.

As example, obesity and associated metabolic diseases are increasing major public health concerns associated with increased mortality. They are characterized by imbalance between energy intake and energy expenditure. Adipose tissues (AT) are among the main organs responsible for energy regulation. While white adipose tissues are specialized in energy storage and release, brown and beige adipose tissues (BAT) dissipate energy as heat thanks to their high mitochondrial content equipped with uncoupling protein-1 (UCP1). Therefore, brown/beige adipocytes are promising cell targets to counteract metabolic diseases and BAT activation has become a main trend in pharmaceutical approach to treat obesity (Mukherjee et al, 2016).

The skilled artisan knows at least three different ways to obtain brown/beige adipocytes in vitro: 1) tissue explant, 2) culture of mature adipocytes, 3) differentiation of adipocytes from precursors cells. As example, WO2017/009263 describe a method of producing brown/beige adipocytes from white adipose tissue cells and/or mesenchymal stroma/stem cells, in particular from subcutaneous white adipose tissue cells, and the use of said brown/beige adipocytes in a cell-based therapy of a subject or in screening platforms. The white adipose tissue cells and/or mesenchymal stroma/stem cells are cultured in a differentiation medium comprising serum, glucocorticoid and a mixture of growth factors to obtain adipogenic progenitor cells. These adipogenic progenitor cells are then brought into contact with one or more adipogenic agents to obtain brown/beige adipocytes. In the Example 2 of WO2017/009263, the adipogenic medium implemented to obtain adipogenic progenitor cells also contains intralipids and forskolin is added to this medium to increase adipocyte browning. It is demonstrated in Muller S, et al. (Sci Rep. 2019), a scientific publication closely related to WO2017/009263, that adding intralipids increases adipogenesis.

When cultured in adequate 3D conditions, stem cells can undergo in-vivo like morphogenesis and turn into self-organized complex structures called organoids. Various avascular and vascular 3D models of white adipose tissue have already been developed. Regarding brown adipose tissue (BAT), main focus was given to the obtention of functional brown/beige adipocytes and not much consideration to other cell compartments. These tissue constructs still suffer from a lack of reproducibility and the failure of long-term culture maintenance (Mehta G et al, 2012). A solution to overcome these limitations is to provide a suitable microenvironment through engineering approaches.

### SUMMARY OF THE INVENTION

The invention is defined by the claims. In particular, the present invention relates to an In vitro use of a TGF-β inhibitor for increasing the viability of a 3D cell aggregate and to an in vitro method for generating a prevascularized 3D cell aggregate comprising the step of contacting mesenchymal stroma/stem cells and endothelial cells with a differentiation medium and a TGF-β inhibitor.

### DETAILED DESCRIPTION OF THE INVENTION

Here, the Inventors describe a new technical solution to improve cell viability and differentiation by implementing techniques conventionally used in the prior art for cell therapy. Surprisingly, the new methods provided herein after can advantageously be implemented without any support matrix or exogenous three-dimensional cell adhesion support. In the present study, the Inventors also provide a modular approach to generate ex vivo a 3D human prevascularized tissue construct, in particular a 3D human prevascularized brown/beige adipose tissue construct. They first developed a brown/beige differentiation medium promoting UCP1 expression and preventing the loss of endothelial cells in 2D culture. Indeed, the Inventors of the present invention surprisingly used a medium closed to the one disclosed in WO2017/009263 and by Muller S, et al. (Sci Rep. 2019) but deprived of intralipids. This medium improves brown/beige adipogenesis while advantageously preserving endothelial cells, as revealed by increase in UCP1 and CD31 expression respectively. This advantage is particularly interesting for generating relevant ex vivo models of adipose tissue, such as prevascularized brown/beige adipocytes-containing 3D cell aggregates,-engineered tissue or organoids as examples. In addition, it should be noted that the addition of adenylate cyclase activator(s) such as forskolin in the medium implemented in the present invention is not mandatory to increase adipocyte browning. The use of a synthetic scaffold is not required either. They then tested the effect of said differentiation medium on 3D spheroid culture. The addition of TGF-β inhibitor in the differentiation medium increases significantly viability, pseudo-vascular formation and adipocyte differentiation.

### In Vitro Method for Producing Highly Viable 3D Cell Aggregates

It is well-known by those skilled in the art that the size of a 3D cell aggregate (correlated to the number of cells) is limited by the capacity of oxygen and nutrients to diffuse to the central cells of said 3D cell aggregate. The Inventors here demonstrate that the use of a TGF-β inhibitor in a culture medium facilitates the survival of 3D cell aggregates. They surprisingly demonstrate that TGF-β inhibition avoid necrotic core and promotes differentiation of stem cells.

Accordingly, in a first aspect, the present invention relates to in vitro use of a TGF-β inhibitor for increasing the viability of a 3D cell aggregate.

As used herein, the term “3D cell aggregate” refers to an aggregation or cluster of cells forming an organized multi-layered structure. Typically, 3D cell aggregate is cultured to allow three-dimensional growth (i.e. 3D culture). In some embodiments, the 3D cell aggregate is a spheroid. As used herein, the term “spheroid” refers to a 3D sphere-like cell aggregate. The skilled artisan well-knows culture systems enabling 3D spheroid formation. Contrarily to 2D culture (e.g. a monolayer), 3D culture is achieved by improving the potential for cells to adhere, as example, using an ultra-low attachment plate. In some embodiments, the methods of the present invention comprise a further step comprising seeding in an ultra-low adherence surface a population of cells. In some embodiments, the 3D cell aggregate is a high-density cell suspension. In some embodiments, a high-density cell suspension is reached when obtaining at least 10³, 10⁴, 10⁵, 10⁶, 10⁷, 10⁸, 10⁹, 10¹⁰, 10¹¹, 10¹², 10¹³, 10¹⁴, 10¹⁵, 10¹⁶ or 10¹⁷ cells per mL in a culture medium. In some embodiments, a high-density cell suspension is reached when obtaining at least 10⁴ cells per mL in a culture medium.

In some embodiments, the 3D cell aggregate is a 3D mesenchymal stroma/stem cell aggregate. In some embodiments, the present invention relates to an in vitro method for increasing the viability of a 3D cell aggregate in a sample wherein the 3D cell aggregate is a 3D mesenchymal stroma/stem cell aggregate, the method comprising the step of contacting said 3D mesenchymal stroma/stem cell aggregate with a TGF-β inhibitor.

As used herein, the term “MSC” or “Mesenchymal Stroma/Stem Cells” refers to multipotent stromal cells having the ability to self-renew and to proliferate in culture. These cells belong to a cell population initially identified in the bone marrow but are present in all tissues. As example, mesenchymal stroma/stem cells can be derived not only from adipose tissue (e.g. after a lipoaspiration or dermolipectomy-herein after named “adipose stem cells”, “adipose tissue-derived stem cells” or “ADSC”) or from bone marrow (herein after named bone marrow-derived mesenchymal stroma/stem cells) of adult human but also from any tissue (muscle, liver, heart . . . ). In some embodiments, the mesenchymal stroma/stem cells are adipose tissue-derived stem cells. In some embodiments, the adipose tissue-derived stem cells are white adipose tissue-derived stem cells (i.e. WAT-derived stem cells). In some embodiments, the mesenchymal stroma/stem cells are infants, child or adult mesenchymal stroma/stem-cells. In some embodiments, the mesenchymal stroma/stem cells are non-embryonic mesenchymal stroma/stem cells.

In some embodiments, the 3D cell aggregate is a 3D cell aggregate wherein the cells are differentiated cells or are cells undergoing differentiation. Accordingly, in some embodiments, the present invention also relates to an in vitro method for increasing the viability of a 3D cell aggregate wherein the 3D cell aggregate is a 3D differentiated cell aggregate or a 3D cell aggregate comprising cells undergoing differentiation, and wherein the method comprises the step of contacting said 3D differentiated cell aggregate or said 3D cell aggregate comprising cells undergoing differentiation with a TGF-β inhibitor.

As example, the 3D cell aggregate may contain, but is not limited to, stem cells, fat cells, endothelial cells, bone cells, blood cells, muscle cells, skin cells, nerve cells, sex cells, pancreatic cells or cancer cells. More particularly, the 3D cell aggregate may contains, but is not limited to, brown adipocytes, brown/beige adipocytes, white adipocytes, endothelial cells, hepatocytes, myocytes, cardiomyocytes, leiomyocytes, rhabdomyocytes, osteoclasts, osteocytes, chondrocytes, pneumocytes, keratocytes, melanocytes, merkel cells, Langerhans cells, alpha cells, beta cells, delta cells, neurons, neuroglial cells, reticulocytes, erythrocytes, enterocytes, neutrophils, eosinophils, basophils, lymphocytes, monocytes or epithelial cells. The 3D cell aggregate may also comprise, but is not limited to, breast cancer cells, uterine/cervical cancer cells, oesophageal cancer cells, pancreatic cancer cells, colorectal cancer cells, kidney cancer cells, ovarian cancer cells, prostate cancer cells, head and neck cancer cells, non-small cell lung cancer cells, stomach cancer cells, tumor cells of mesenchymal origin (i.e; sarcoma, fibrosarcoma and rhabdomyoscarcoma) tumor cells of the central and peripheral nervous system (i.e; including astrocytoma, neuroblastoma, glioma, glioblatoma) or thyroid cancer cells.

In some embodiments, the 3D cell aggregate comprises 1, 2, 3, 4, 5, 6, 7, 8, 9 or 10 different types of cells. In some embodiments, the 3D cell aggregate comprises mesenchymal stroma/stem cells and differentiated cells. In some embodiments, the 3D cell aggregate comprises mesenchymal stroma/stem cells and endothelial cells.

In some embodiments, the 3D cell aggregate is a prevascularized 3D cell aggregate. In some embodiments, the 3D cell aggregate is a prevascularized 3D cell aggregate comprising mesenchymal stroma/stem cells. In some embodiments, the 3D cell aggregate is a prevascularized 3D cell aggregate comprising differentiated cells.

As used herein, the term “prevascularized 3D cell aggregate” refers to an aggregation or cluster of cells forming an organized multi-layered structure and containing endothelial cells assembled to form an organized endothelial network among said cells, hereby generating a preformed vasculature.

As used herein, the term “viability” refers to an assessment of the proportion of living, healthy cells within a population of cells. Cell viability assays provide a readout through, as example, the measurement of metabolic activity, ATP content or cell proliferation. More particularly, cell viability may be assessed by 3D cell aggregate size measurements, iodure propidium staining, DNA quantification, histology, immunohistochemistry, or immunofluorescence.

As used herein, the term “TGF-β” has its general meaning in the art and refers to the Transforming Growth Factor-β which is a multifunctional cytokine belonging to the TGF superfamily. The TGF-β includes three different isoforms, namely TGF-β1, TGF-β2 and TGF-β3.

As used herein, the term “TGF-β inhibitor” refers to a compound able to inhibits TGF-β gene expression or able to inhibit the activity of TGF-β (e.g. interactions with other partners). Such inhibitors are well-known in the art and comprise low molecular weight compounds, antibodies directed against TGF-β, single domain antibodies directed against TGF-β, aptamers, polypeptides such as a mutated TGF-β proteins or similar proteins without the function of TGF-β, inhibitors of TGF-β gene expression, small inhibitory RNAs (siRNAs), small double stranded RNA (dsRNA) or ribozymes. Thus, in some embodiments, the TGF-β inhibitor is a low molecular weight compound, an antibody directed against TGF-β, a single domain antibody directed against TGF-β, an aptamer, a polypeptide, an inhibitor of TGF-β gene expression, a small inhibitory RNA (siRNA), a small double stranded RNA (dsRNA) or a ribozyme.

In some embodiments, the TGF-β inhibitor is a low molecular weight compound, e.g. a small organic molecule (natural or not). The term “small organic molecule” refers to a molecule (natural or not) of a size comparable to those organic molecules generally used in pharmaceuticals. The term excludes biological macromolecules (e.g., proteins, nucleic acids, etc.). Preferred small organic molecules range in size up to about 10000 Da, more preferably up to 5000 Da, more preferably up to 2000 Da and most preferably up to about 1000 Da. In some embodiments, the TGF-β inhibitor is SB431542. As used herein, the term “SB431542” refers to 4-[4-(1,3-benzodioxol-5-yl)-5-(2-pyridinyl)-1H-imidazol-2-yl]benzamide (CAS number: 301836-41-9). In some embodiments, the TGF-β inhibitor is added in an amount between 1 μg·mL⁻¹ and 10 μg·mL⁻¹, preferentially 5 μg·mL^{-1.}

### In Vitro Method for Producing Prevascularized 3D Cell Aggregates

In a second aspect, the present invention relates to an in vitro method for generating a prevascularized 3D cell aggregate comprising the step of contacting mesenchymal stroma/stem cells and endothelial cells with a differentiation medium and a TGF-β inhibitor.

In some embodiments, the mesenchymal stroma/stem cells are adipose tissue-derived stroma/stem cells. In some embodiments, the adipose-tissue derived stroma/stem cells are white adipose tissue-derived stroma/stem cells.

As used herein, the term “Adipose Stroma/Stem Cells” or “Adipose tissue-derived Stroma/Stem Cells” or “ASC” refers to Mesenchymal Stroma/Stem Cells (MSC) originating from adipose tissue.

As used herein, the term “Endothelial Cells” or “EC” refers to a population of cells lining the walls of vessels, tightly connected to each other by cell-cell junctions. The endothelium is a thin membrane that lines the inside of the heart and blood vessels. Endothelial cells release substances that control vascular relaxation and contraction as well as enzymes that control blood clotting, immune function and platelet adhesion. EC can be identified by their cell surface biomarkers, and are in particular CD31+. Endothelial Progenitor Cells (EPC) are able to differentiate into endothelial cells.

The mesenchymal stroma/stem cells and the endothelial lineage cells implemented in the method of the invention may be obtained from primary cells such as, for example, human dermal microvascular endothelial cells (hDMEC) and brown adipocytes progenitors and/or precursors (e.g. hiPSC-BA). Alternatively, these cells may be obtained as cellular sample derived from white adipose tissue, such as Stromal Vascular Fraction (SVF). In some embodiments, the mesenchymal stroma/stem cells and endothelial cells are derived from a Stromal Vascular Fraction (SVF).

As used herein, the term “Stromal Vascular Fraction” or “SVF” corresponds to a heterogeneous cell population derived from adipose tissues that contains typically adipose-derived stem cells (ADSC), endothelial lineage cells, macrophages, dendritic cells and lymphocytes. In some embodiments, the mesenchymal stroma/stem cells are collected from a SVF derived from a dermolipectomy or a lipoaspirate (i.e. an ex-vivo waste product of liposuction). Typically, when cells are isolated from adipose tissue extracellular matrix by enzymatic digestion, the adipocytes float in the cell suspension and are eliminated. About 90% of the volume of adipose tissue is made up of adipocytes, the remaining 10% represents the stromal vascular fraction. In some embodiments, the mesenchymal stroma/stem cells and endothelial cells are derived from a SVF collected on a subject and are destinated to be relocated in said subject, particularly when differentiated in a prevascularized 3D cell aggregates, more particularly when differentiated in a prevascularized brown/beige adipocytes-containing 3D cell aggregates. Thus, in some embodiments, the prevascularized 3D cell aggregate is an autologous 3D cell aggregate. In some embodiments, the prevascularized brown/beige adipocytes-containing 3D cell aggregate is an autologous brown/beige adipocytes-containing 3D cell aggregate. By using autologous 3D cell aggregate or autologous brown/beige adipocytes-containing 3D cell aggregate in a treatment, immunological tolerance and safety are favoured, particularly by promoting the inhibition of regulatory cells and by decreasing ongoing inflammation.

As used herein, the term “subject” refers to a mammal, such as rodent, a feline, a canine or a primate. In some embodiments the subject is a human. In some embodiments, the subject is a mouse.

In some embodiments, the in vitro method comprises the step of contacting said mesenchymal stroma/stem cells (MSC) and endothelial cells with a differentiation medium, a TGF-β inhibitor and no exogenous free fatty acids. In some embodiments, the exogenous free fatty acids are intralipids.

As used herein, the term “differentiation medium” refers to a medium formulated to optimize the preferential differentiation of immature cells (e.g. MSC or ASC) into mature cells with specific functions (e.g. adipocytes or brown/beige adipocytes). As example, when the differentiation medium comprises an adipogenic agent, the differentiation medium is able to induce the differentiation of mesenchymal stroma/stem cells into adipocytes. When the differentiation medium comprises a browning agent, the differentiation medium is able to induce the differentiation of mesenchymal stroma/stem cells into brown/beige adipocytes.

As used herein, the term “adipogenic medium” refers to a medium comprising at least one adipogenic agent. As used herein, the term “adipogenic agent” refers to a compound able to induce the differentiation of mesenchymal stroma/stem cells into adipocytes. Preferentially, the adipogenic agent is a browning agent, able to induce the differentiation of mesenchymal stroma/stem cells into brown/beige adipocytes. Thus, in some embodiments, the differentiation medium comprises at least one adipogenic agent. In some embodiments, the differentiation medium comprises at least one browning agent. As example, brown/beige adipogenesis can be revealed by UCP1 increased expression (i.e. Uncoupling Protein 1; Entrez Gene: 7350). In some embodiments, the UCP1 increased expression is revealed by an UCP1 inducer. In some embodiments, the browning agent is a Bone Morphogenic Protein (BMP). In some embodiments, the browning agent is BMP7.

As used herein, the term “Bone Morphogenic Protein” or “BMP” refers to a bone-derived factors capable of inducing ectopic bone formation and belonging to the Transforming Growth Factor (TGF-β) superfamily. Examples of BMPs include but are not limited to BMP1, BMP2, BMP3, BMP4, BMP5, BMP6, BMP7, BMP8 and BMP9. In some embodiments, the BMP is BMP7. In some embodiments, Bone Morphogenic Protein (BMP) is added in an amount between 20 ng·mL⁻¹ and 80 ng·mL⁻¹, preferably 50 ng·mL⁻¹.

In some embodiments, the differentiation medium comprises α-MEM, a mix of growth factors, insulin or insulin-like growth factor, apotransferrin and a bone morphogenic protein. In some embodiments, the differentiation medium comprises α-MEM, a mix of growth factor, insulin or insulin-like growth factor, apotransferrin, a bone morphogenic protein and no exogenous free fatty acids. In some embodiments, the differentiation medium consists essentially in α-MEM, a mix of growth factor, insulin or insulin-like growth factor, apotransferrin and a bone morphogenic protein. In some embodiments, the differentiation medium consists essentially in α-MEM, a mix of growth factor, insulin or insulin-like growth factor, apotransferrin, a bone morphogenic protein and no exogenous free fatty acids. As used herein, the term “consist essentially in” means that the differentiation medium does not comprise any other active substance that has an effect on the differentiation of stem cells into differentiated cells, in particular adipose stem cell into brown/beige adipocytes. In some embodiments, the differentiation medium consists in α-MEM, a mix of growth factor, insulin or insulin-like growth factor, apotransferrin and a bone morphogenic protein. In some embodiments, the differentiation medium consists in α-MEM-ASP, foetal bovine serum, insulin, apotransferrin and BMP7.

As used herein, the term “α-MEM” refers to α-Minimal Essential Medium. In some embodiments, α-MEM comprises amino acids, sodium pyruvate, lipoic acid, vitamin B12, biotin and/or ascorbic acid. In some embodiments, α-MEM consists essentially in amino acids, sodium pyruvate, lipoic acid, vitamin B12, biotin and ascorbic acid. In some embodiments, α-MEM consists in amino acids, sodium pyruvate, lipoic acid, vitamin B12, biotin and ascorbic acid. In some embodiments, the α-MEM is supplemented with amphotericin B and streptomycin/penicillin (ASP). In some embodiments, the α-MEM is supplemented with amphotericin B in an amount between 0.05% (v/v) and 1.5% (v/v), preferentially 0.1% (v/v) and streptomycin/penicillin in an amount between 0.05% (v/v) and 1.5% (v/v), preferentially 1% (v/v) (ASP). In some embodiments, the α-MEM is α-MEM-ASP.

As used herein, the term “mix of growth factor” refers to a growth supplement for cell culture media containing at least two growth-promoting factors. In some embodiments, the mix of growth factor comprises at least two growth-promoting factors selected in the group comprising growth factor of the VEGF family, of the EGF family, of the FGF family or an insulin-like growth factor. In some embodiments, the mix of growth factor comprises at least two growth-promoting factors selected in the group consisting of growth factor of the VEGF family, of the EGF family, of the FGF family or an insulin-like growth factor. In some embodiments, the mix of growth factor is provided by serum. In some embodiments, the mix of growth factor is provided by bovine foetal serum. In some embodiments, the mix of growth factor is provided by platelet lysate. For in vitro purposes, foetal bovine serum may be preferred. For in vivo purposes, platelet lysate may be preferred. In some embodiments, the mix of growth factor is added in an amount between 1% (v/v) and 5% (v/v), preferably 2% (v/v).

As used herein, the term “insulin” refers to a peptide hormone produced by β-cells of the pancreatic islets encoded in humans by INS gene (Entrez Gene: 3630). Alternatively, Insulin-like Growth Factor (IGF) may be used to substitute for insulin. As example, IGF may be selected from the group consisting of IGF-1, IGF-2, IGFL1, IGFL2, IGFL3 and IGFL4. Synthetic analogs can also be used. In some embodiments, insulin or Insulin-like Growth Factor (IGF) is added in an amount between 1 μg·mL⁻¹ and 10 μg·mL⁻¹, preferably 5 μg·mL⁻¹.

As used herein, the term “apotransferrin” refers to an iron free transferrin. Transferrin are single chain glycoproteins with two nonidentical iron-binding sites having a high affinity for ferric iron under physiological conditions. Transferrins are found in vertebrates and mediate the transport of iron through blood plasma. When not bound to iron, transferrin is known as “apotransferrin”. In some embodiments, apotransferrin is added in an amount between 5 μg·mL⁻¹ and 15 μg·mL⁻¹, preferably 10 μg·mL¹.

As used herein, the term “free fatty acid” refers to a carboxylic acid with an aliphatic chain which is either saturated or unsaturated. As used herein, the term “exogenous free fatty acid” refers to free fatty acids added in a culture medium and originating from outside of the 3D cell aggregate. In some embodiments, the differentiation medium is deprived of intralipids® (CAS: 68890-65-3). Intralipid® is a lipid emulsion comprising soybean oil, glycerin and phospholipids. Intralipid® is source of fats, and in particular of exogenous free fatty acids.

In some embodiments, the present methods of the invention comprise a further step of contacting mesenchymal stroma/stem cells and endothelial cells, or SVF comprising mesenchymal stroma/stem cells and endothelial cells, with an amplification medium to generate an amplified population of cells.

As used herein, the term “amplification medium” refers to a medium able to optimize the growth and proliferation of cells. In some embodiments, the amplification medium comprises serum and growth factors. In some embodiments, the amplification medium is an EGM2 medium. In some embodiments, the amplification medium comprises α-MEM and EGM2 medium supplemented with amphotericin B and streptomycin/penicillin. In some embodiments, the amplification medium consists in EGM2 medium supplemented with amphotericin B and streptomycin/penicillin. In some embodiments, the amplification medium consists in α-MEM and EGM2 medium supplemented with amphotericin B and streptomycin/penicillin. In some embodiments, the amplification medium comprises Vascular Endothelial Growth Factor (VEGF). In some embodiments, VEGF may be selected from the group consisting of VEGF-A, preferably VEGF-A splice form VEGF121, VEGF 145 or VEGF 165, VEGF-B, VEGF-C, VEGF-D and PGF. In some embodiments, VEGF is added in an amount between 0.4 ng·mL⁻¹ and 0.6 ng·mL⁻¹, preferably 0.5 ng·mL⁻¹. In some embodiments, the amplification medium comprises VEGF and Insulin-like Growth Factor (IGF). In some embodiments, the IGF may be selected from the group consisting of IGF-1, IGF-2, IGFL1, IGFL2, IGFL3 and IGFL4 and synthetic analogs thereof such as long(R3)-IGF-1. In some embodiments, Insulin-like Growth Factor (IGF) is added in an amount between 15 ng·mL⁻¹ and 25 ng·mL⁻¹, preferably 20 ng·mL⁻¹. In some embodiments, the amplification medium comprises serum, IGF and VEGF. In some embodiments, serum is added in an amount between 1% (v/v) and 3% (v/v), preferably 2% (v/v). In some embodiments, the amplification medium comprises VEGF, long(R3)-IGF-1 and foetal bovine serum. In some embodiments, the amplification medium consists in VEGF, long(R3)-IGF-1, foetal bovine serum, EGF, FGF, heparin, ascorbic acid, hydrocortisone and gentamicin. In some embodiments, EGF is added in an amount between 4 ng·mL⁻¹ and 6 ng·mL⁻¹, preferably 5 ng·mL⁻¹. In some embodiments, FGF is added in an amount between 15 ng·mL⁻¹ and 25 ng·mL⁻¹, preferably 20 ng·mL⁻¹. In some embodiments, heparin is added in an amount between 15 μg·mL⁻¹ and 25 μg·mL⁻¹, preferably 22.5 μg·mL⁻¹. In some embodiments, ascorbic acid is added in an amount between 0.5 μg·mL⁻¹ and 1.5 μg·mL⁻¹, preferably 1 μg·mL⁻¹. In some embodiments, hydrocortisone is added in an amount between 0.1 μg·mL⁻¹ and 0.3 g·mL⁻¹, preferably 0.2 μg·mL⁻¹. In some embodiments, gentamicin is diluted 1/1000 in said amplification medium. In some embodiments, the amplification medium consists in α-MEM, VEGF, long(R3)-IGF-1, foetal bovine serum, EGF, FGF, heparin, ascorbic acid, hydrocortisone, gentamicin, amphotericin B and streptomycin/penicillin. In some embodiments, the amplification medium consists in α-MEM, endothelial basal medium, VEGF, long(R3)-IGF-1, foetal bovine serum, EGF, FGF, heparin, ascorbic acid, hydrocortisone, gentamicin, amphotericin B and streptomycin/penicillin. Typically, in some embodiments, the culture of the stem cells or cells in said amplification medium, is carried out for about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

In the context of the invention, an amplified population of SVF cells is named “P0-SVF”. P0-SVF contains typically mesenchymal stroma/stem cells, endothelial cells and macrophages. The main advantage of using P0-SVF rather than SVF lies in selection by adhesion which rapidly eliminates the majority of immune cells and greatly enriches in progenitor cells. It is therefore possible, with the same sample, to obtain many more cells of interest. It results in a greater homogeneity of the cells, and therefore a better response to inducers. Thus, in some embodiments, the present methods of the invention comprise a further step of contacting P0-SVF comprising mesenchymal stroma/stem cells and endothelial cells with an amplification medium to generate an amplified population of cells. In some embodiments, the population is amplified until reaching at least 70% of confluency in a culture medium. In some embodiments, the population is amplified until reaching at least 80% of confluency in a culture medium. In some embodiments, the population is amplified until reaching at least 90% of confluency in a culture medium. In some embodiments, the population is amplified until reaching 100% of confluency in a culture medium. In some embodiments, the population is amplified until reaching at least 10³, 10⁴, 10⁵, 10⁶, 10⁷, 10⁸, 10⁹, 10¹⁰, 10¹¹, 10¹², 10¹³, 10¹⁴, 10¹⁵, 10¹⁶ or 10¹⁷ cells per mL in a culture medium.

Accordingly, in some embodiments, the present invention relates to an in vitro method for generating a prevascularized 3D cell aggregate comprising the steps of:
Contacting a SVF comprising mesenchymal stroma/stem cells and endothelial cells with an amplification medium to generate an amplified population of cells;
Seeding said amplified population of cells in an ultra-low adherence surface; and
Contacting said seeded amplified population of cells with a differentiation medium and a TGF-β inhibitor.

In some embodiments, the present invention relates to an in vitro method for generating a prevascularized 3D cell aggregate comprising the steps of:
Contacting a SVF comprising mesenchymal stroma/stem cells and endothelial cells with an amplification medium to generate an amplified population of cells;
Seeding said amplified population of cells in an ultra-low adherence surface; and
Contacting said seeded amplified population of cells with a differentiation medium and a TGF-β inhibitor, said differentiation medium being deprived of intralipids.

In some embodiments, the present invention relates to an in vitro method for generating a prevascularized brown/beige adipocytes-containing 3D cell aggregate comprising the steps of:
Contacting a SVF comprising mesenchymal stroma/stem cells and endothelial cells with an amplification medium to generate an amplified population of cells;
Seeding said amplified population of cells in an ultra-low adherence surface; and
Contacting said seeded amplified population of cells with a differentiation medium comprising α-MEM, a mix of growth factor, insulin, apotransferin, a bone morphogenic protein and a TGF-β inhibitor,

Whereby the 3D cell aggregate thus generated is a prevascularized brown/beige adipocytes-containing 3D cell aggregate.

In some embodiments, the present invention relates to an in vitro method for generating a prevascularized brown/beige adipocytes-containing 3D cell aggregate comprising the steps of:
Contacting a SVF comprising mesenchymal stroma/stem cells and endothelial cells with an amplification medium to generate an amplified population of cells;
Seeding said amplified population of cells in an ultra-low adherence surface; and
Contacting said seeded amplified population of cells with a differentiation medium comprising α-MEM, a mix of growth factor, insulin, apotransferin, a bone morphogenic protein and a TGF-β inhibitor, said differentiation medium being deprived of intralipids, Whereby the 3D cell aggregate thus generated is a prevascularized brown/beige adipocytes-containing 3D cell aggregate.

In some embodiments, the present invention relates to an in vitro method for generating a prevascularized brown/beige adipocytes-containing 3D cell aggregate comprising the steps of:
Contacting a SVF comprising mesenchymal stroma/stem cells and endothelial cells with an amplification medium to generate an amplified population of cells;
Seeding said amplified population of cells in an ultra-low adherence surface; and
Contacting said seeded amplified population of cells with a differentiation medium consisting in α-MEM-ASP, foetal bovine serum, insulin, apotransferin, BMP7 and SB431542

whereby the 3D cell aggregate thus generated is a prevascularized brown/beige adipocytes containing 3D cell aggregate.

As used herein, the term “brown/beige adipocytes-containing 3D cell aggregate” refers to an aggregation or cluster of brown/beige adipocytes forming an organized multi-layered structure. In some embodiments, the brown/beige adipocytes-containing 3D cell aggregate is a spheroid. In some embodiments, the brown/beige adipocytes-containing 3D cell aggregate is a high-density cell suspension. In some embodiments, a high-density cell suspension is reached when obtaining at least 10³, 10⁴, 10⁵, 10⁶, 10⁷, 10⁸, 10⁹, 10¹⁰, 10¹¹, 10¹², 10¹³, 10¹⁴, 10¹⁵, 10¹⁶ or 10¹⁷ cells per mL in a culture medium. In some embodiments, a high-density cell suspension is reached when obtaining at least 10⁴ cells per mL in a culture medium.

As used herein, the term “prevascularized brown/beige adipocytes-containing 3D cell aggregate” refers to a cluster or aggregation of brown/beige adipocytes-containing endothelial cells self-assembling to form organized endothelial networks among brown/beige adipocytes. Native human subcutaneous adipose tissue is organized into unilocular or multilocular adipocytes interspersed within a dense vascularization. Thus, obtaining densely prevascularized brown/beige adipocytes-containing 3D cell aggregate is reminiscent of the cellular architecture found in vivo.

As used herein, the term “adipocytes” refers to a type of cells specialized in the storage and release of lipids in adipose tissue, an organ specialized in storing and releasing energy in the form of triglycerides. Adipocytes are classified as white, beige or brown adipocytes. White adipocytes have a white adipokine secretory function with a morphology characterized by the presence of large lipid vacuoles. Brown adipocytes are responsible for thermogenesis with expression of the Uncoupling Protein 1 (UCP1; Entrez Gene: 7350) gene within the mitochondrial membrane, with a morphology characterized by the presence of several small lipid vacuoles, a high content of mitochondria and the secretion of brown adipokines. Adipose tissue constitutes the only reserve of energy that can be mobilized in the long term and therefore occupies a preponderant place in the control of the energy balance in mammals. Consequently, a defect in the storage of lipids within the adipose tissue leads to significant metabolic disorders.

### Prevascularized 3D Cell Aggregate and Their Uses

A third aspect of the invention relates to a prevascularized 3D cell aggregate obtained with the in vitro methods as previously described. In some embodiments, the 3D cell aggregate is a prevascularized brown/beige adipocytes-containing 3D cell aggregate.

### Metabolic Disorders and Tissue Transplantation

In another aspect, the present invention relates to at least one prevascularized 3D cell aggregate generated with the methods of the invention for use in the treatment of a metabolic disorder.

As used herein, the term “metabolic disorder” denotes a state that negatively alters the body's processing and distribution of macronutrients such as proteins, fats and carbohydrates. It occurs when abnormal chemical reactions in the body alter the normal metabolic process. As example, metabolic disorders include type 2 diabetes, impaired glucose tolerance, obesity, insulin resistance, dyslipidemia, non-alcoholic hepatic syndrome (NASH), hypertension and cardiovascular diseases. Thus, in some embodiments, the metabolic disorder is selected from the group consisting of type 2 diabetes, impaired glucose tolerance, obesity, insulin resistance, dyslipidemia, non-alcoholic hepatic syndrome (NASH), hypertension and cardiovascular diseases.

As used herein, the terms “treating”, “treatment” or “therapy” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. In some embodiments, the term “treatment” particularly refers to the preventive treatment of a metabolic disorder, in particular with a cell based-therapy comprising at least one prevascularized 3D cell aggregates, in particular at least one prevascularized brown/beige adipocytes-containing 3D cell aggregates. The treatment may be administered to a subject having a medical disorder or a subject likely to suffer from the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of a cell based-therapy to a subject during the initial period of a treatment regimen. An induction regimen may employ (in part or in whole) a “loading regimen”, which may include administering a greater dose of the cell based-therapy than a physician would employ during a maintenance regimen, administering a cell based-therapy more frequently than a physician would administer the cell based-therapy during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a cell based-therapy at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the terms “administering” or “administration” refer to the act of injecting or otherwise physically delivering a substance as it exists outside the body (e.g., at least one prevascularized 3D cell aggregate or at least one prevascularized brown/beige adipocytes-containing 3D cell aggregate) into the subject, such as by mucosal, intradermal, subcutaneous delivery and/or any other method of physical delivery described herein or known in the art. When a disease, or a symptom thereof, is being treated, administration of the at least one prevascularized 3D cell aggregate or the at least one prevascularized brown/beige adipocytes-containing 3D cell aggregate typically occurs after the onset of the disease or symptoms thereof. When a disease or symptoms thereof, are being prevented, administration of the at least one prevascularized 3D cell aggregate or the at least one prevascularized brown/beige adipocytes-containing 3D cell aggregate typically occurs before the onset of the disease or symptoms thereof.

As used herein, the term “efficient” denotes a state wherein the administration of at least one prevascularized 3D cell aggregate or at least one prevascularized brown/beige adipocytes-containing 3D cell aggregate to a subject permits to prevent, cure, delay the onset of, reduce the severity of, or ameliorate one or more symptoms of a disorder or recurring disorder, or to prolong the survival of a subject beyond that expected in the absence of such treatment. A “therapeutically effective amount” is intended for a minimal amount of cell-based therapy which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; at least one prevascularized 3D cell aggregate or at least one prevascularized brown/beige adipocytes-containing 3D cell aggregate used in combination or coincidental with another specific compound employed; and like factors well known in the medical arts. For example, it is well-known within the skill of the art to start doses of the cell-based therapy at levels lower than those required to achieve the desired therapeutic effect and to gradually increase the dosage until the desired effect is achieved.

In some embodiments, the present invention relates to a method of treating a subject suffering from a metabolic disorder comprising administering to said subject a therapeutically effective amount of at least one prevascularized 3D cell aggregate. In some embodiments, the present invention relates to a method of treating a subject suffering from a metabolic disorder comprising administering to said subject a therapeutically effective amount of at least one prevascularized brown/beige adipocytes-containing 3D cell aggregate.

In some embodiments, the present invention relates to at least one prevascularized 3D cell aggregate generated with the methods of the present invention for use in tissue transplantation. In some embodiments, the at least one prevascularized 3D cell aggregate is a prevascularized brown/beige adipocytes-containing 3D cell aggregate. Thus, in another embodiment, the present invention relates to at least one prevascularized brown/beige adipocytes-containing 3D cell aggregate generated with the methods of the present invention for use in adipose-tissue transplantation. In some embodiments, the adipose-tissue transplantation is an autologous tissue transplantation.

As used herein, the term “autologous tissue transplantation” denotes a procedure in which a subject's own tissue is collected to replace or sustain the activity of his damaged tissue.

The prevascularized 3D cell aggregate can be introduced in a therapeutic composition for use in tissue transplantation. As example, a prevascularized brown/beige adipocytes-containing 3D cell aggregate can be introduced in a therapeutic composition for use in adipose-tissue transplantation.

### Metabolic Disorders and Therapeutic Compositions

In another aspect, the present invention relates to a therapeutic composition comprising at least one prevascularized 3D cell aggregate generated with the methods of the invention. In some embodiments, the invention relates to a therapeutic composition comprising at least one prevascularized 3D cell aggregate generated with the methods of the invention for use in the tissue transplantation and/or in the treatment of a metabolic disorder in a subject in need thereof. In some embodiments, the at least one prevascularized brown/beige adipocytes-containing 3D cell aggregate is a prevascularized brown/beige adipocytes-containing 3D cell aggregate generated with the methods of the invention. In some embodiments, the invention relates to a therapeutic composition comprising at least one prevascularized brown/beige adipocytes-containing 3D cell aggregate generated with the methods of the invention for use in the adipose-tissue transplantation and/or in the treatment of a metabolic disorder in a subject in need thereof. Preferably, the therapeutic compositions contain vehicles which are pharmaceutically acceptable for a formulation capable of being injected or transplanted. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The doses used for the administration can be adapted as a function of various parameters, and in particular as a function of the mode of administration used, of the relevant pathology, or alternatively of the desired duration of treatment. Pharmaceutical compositions of the present invention may comprise a further therapeutic active agent.

### Method of Screening Compounds That Modulates Tissue Activity

In another aspect, the prevascularized 3D cell aggregate generated with the methods of the invention can be as example used as a search tool in an in vitro method of screening a compound that modulates tissue activity. In some embodiments, a prevascularized brown/beige adipocytes-containing 3D cell aggregate generated with the methods of the invention is used as a search tool in an in vitro method of screening compounds that modulates brown/beige adipose tissue activity.

Accordingly, in some embodiments, the present invention relates to an in vitro method of screening a compound that modulates tissue activity comprising contacting the prevascularized 3D cell aggregate generated with the methods of the invention with a compound suspected to modulate tissue activity and monitoring the effect of said compound on the activity of said prevascularized 3D cell aggregate. In some embodiments, the present invention relates to an in vitro method of screening a compound that modulate brown/beige adipose tissue activity comprising contacting the prevascularized brown/beige adipocytes-containing 3D cell aggregate generated with the methods of the invention with a compound suspected to modulate adipose tissue activity and monitoring the effect of said compound on the activity of said prevascularized brown/beige adipocytes-containing 3D cell aggregate.

In a more particular embodiment, the in vitro method of screening a compound that modulate tissue activity comprises the steps of:
Measuring the level of at least one marker into said prevascularized 3D cell aggregate generated with the methods of the invention;
Contacting the prevascularized 3D cell aggregate generated with the methods of the invention with said compound suspected to modulate tissue activity;
Measuring the level of said at least one marker into said prevascularized 3D cell aggregate generated with the methods of the invention;
Comparing the level measured before the contact of said compound suspected to modulate tissue activity with the level measured after the contact of said compound suspected to modulate tissue activity or with a predetermined reference value;
Concluding that the compound suspected to modulate tissue activity has an effect or not on said tissue activity depending on the evolution of the level of said marker.

In an even more particular embodiment, the in vitro method is an in vitro method of screening a compound that modulates brown/beige adipose tissue activity comprises the steps of:
Measuring the level of at least one marker into a prevascularized brown/beige adipocytes-containing 3D cell aggregate generated with the methods of the invention;
Contacting the prevascularized brown/beige adipocytes-containing 3D cell aggregate generated with the methods of the invention with a compound suspected to modulate adipose tissue activity;
Measuring the level of said at least one marker into said prevascularized brown/beige adipocytes-containing 3D cell aggregate generated with the methods of the invention;
Comparing the level measured before the contact of said compounds suspected to modulate adipose tissue activity with the level measured after the contact of said compounds suspected to modulate adipose tissue activity or with a predetermined reference value;
Concluding that the compound suspected to modulate adipose tissue activity has an effect or not on said brown/beige adipose tissue activity depending on the evolution of the level of said marker.

In some embodiments, the predetermined reference value is relative to a number or value derived from studies, as example, led on cells or tissues of subjects, including without limitation, subjects of the same or similar age range, subjects in the same or similar ethnic group, and subjects having the same severity of lesion. Such predetermined reference values can be derived from statistical analyses and/or risk prediction data obtained from mathematical algorithms and computed indices.

Said measurement of the level of at least one marker may involve as example intercalating agents or fluorescent dyes in techniques well known to those skilled in the art. As example, said level can be measured at the transcriptomic or protein level.

In some embodiments, the marker is a survival marker assessing cell viability (e.g. propium iodide). In some embodiments, the marker is an adipogenesis marker (e.g. Adipoq, PPARg, FABP4). In some embodiments, the marker is brown/beige adipocytes marker (e.g. UCP1, CIDEA, PGC1a).

In a particular embodiment, the in vitro methods of screening a compound that modulates brown/beige adipose tissue activity is used for screening compounds increasing the expression of mitochondrial activity in said prevascularized brown/beige adipocytes-containing 3D cell aggregate. In another particular embodiment, the in vitro methods of screening a compound that modulates brown/beige adipose tissue activity is used for screening a compound increasing UCP1 expression in said prevascularized brown/beige adipocytes-containing 3D cell aggregate. Thus, in some embodiments, the in vitro method comprises a further step consisting in monitoring UCP1 expression.

### Medium and Kit for Generating a Prevascularized 3D Cell Aggregate

### Medium for Generating a Prevascularized 3D Cell Aggregate

In another aspect, the present invention relates to a medium for producing prevascularized 3D cell aggregate.

In some embodiments, the medium for producing a prevascularized brown/beige adipocytes-containing 3D cell aggregate comprises at least one browning agent and a TGF-β inhibitor. In some embodiments, the medium for producing a prevascularized brown/beige adipocytes-containing 3D cell aggregate comprises BMP 7 and a TGF-β inhibitor. Said medium can be defined as a differentiation medium. In some embodiments, said medium is deprived of intralipids.

In some embodiments, the medium comprises α-MEM, a mix of growth factors, insulin or insulin-like growth factor, apotransferrin, a bone morphogenic protein and a TGF-β inhibitor. In some embodiments, the medium consists in α-MEM, a mix of growth factors, insulin or insulin-like growth factor, apotransferrin, a bone morphogenic protein and a TGF-β inhibitor. In some embodiments, the medium consists essentially in α-MEM, a mix of growth factors, insulin or insulin-like growth factor, apotransferrin, a bone morphogenic protein and a TGF-β inhibitor. In some embodiments, the medium comprises α-MEM-ASP, foetal bovine serum, insulin, apotransferin, BMP7 and SB431542. In some embodiments, the medium consists essentially in α-MEM-ASP, foetal bovine serum, insulin, apotransferrin, BMP7 and SB431542. In some embodiments, the medium consists in α-MEM-ASP, foetal bovine serum, insulin, apotransferrin, BMP7 and SB431542.

### Kit for Generating a Prevascularized 3D Cell Aggregate

In another aspect, the present invention relates to a kit for producing a prevascularized 3D cell aggregate comprising the medium for generating a prevascularized 3D cell aggregate and at least one selected from the group consisting of an amplification medium and an ultra-low adherence surface. In some embodiments, the present invention related to a kit for producing prevascularized brown/beige adipocytes-containing 3D cell aggregate comprising the medium for generating a prevascularized 3D cell aggregate and at least one selected from the group consisting of an amplification medium and an ultra-low adherence surface. In some embodiments, the kit comprises further comprises an amplification medium and an ultra-low adherence surface.

### FIGURES

FIG. 1. Development of a brown adipogenic cocktail that preserves endothelial cells from P0-SVF cells. 2D culture of P0-SVF cells were maintained for 21 days of differentiation either in standard white adipogenic cocktail (STD), adipogenic cocktail 1 containing intralipids (C1), adipogenic cocktail 2 (C2) corresponding to C1 without intralipids. (A) Gene expression analysis of UCP/brown adipocytes markers, ADIPOQ generic adipocyte marker, and CD31 endothelial cell marker. Folds are relatively expressed to non-differentiated cells at day 0 of differentiation. Data are expressed as mean +/−standard deviation of three independent experiments from different human donors. Statistical analysis was performed by ANOVA-1 type III followed by post-hoc tukey's test on AACT values. *p<0.05, **p<0.01, ***p<0.001. (B) Immunofluorescence images of adipocytes and pseudo-vascular organization at day 21 of differentiation. Lipids-containing cells were stained with bodipy while endothelial cells and smooth muscle like cells were revealed by CD31 and aSMA stainings respectively. White squared image is a zoomed area showing endothelial cells organization aligned with aSMA+cells in between areas containing differentiated adipocytes with P0-SVF cells cultivated in C2 adipogenic cocktail. Scales: 200 μm.

FIG. 2. TGF-βinhibition promotes cell maintenance, adipocyte differentiation and vascular formation of 3D spheroids from P0-SVF cells. (A) Evolution of spheroid size with differentiation duration in C2 medium in absence (lower curve, n=9) or in presence of SB431542 (referred as C2+SB4 condition), a potent TGF-β inhibitor, (upper curve, n=9). Photos show representative brightfield images of spheroids before and after differentiation in each media. Statistical analysis was performed by two ways ANOVA followed by post-hoc tukey's multiple comparisons (#: DO vs Dx time point for each culture media, *: C2 vs C2+SB4). (B) Average DNA content per spheroid before (n=6) and after differentiation under C2 (n=6) or C2+SB4 condition (n=6). (C) Immunofluorescence images of propidium iodide (dead cells) and DAPI (all cell nuclei) stainings of P0-SVF spheroids under C2 or C2+SB4 condition. (D) Quantification of average propidium iodide positive cells percentage within spheroids at day 21 in C2 (n=4) or C2+SB4 (n=4) media. (E) Gene expression analysis of brown adipocytes markers (UCP1, CIDEA, PGC1a), generic adipocyte markers (PPARg2, FABP4, ADIPOQ) and myofibroblast markers (aSMA, COL1a, CTGF) in spheroids after 21 days of differentiation in C2 (n=8) or C2+SB4 (n=8) media. Folds are expressed relatively to undifferentiated spheroids. (F) Immunofluorescence images of adipocytes and pseudo-vascular organization within P0-SVF spheroids at day 21 in C2 or C2+SB4 media. Lipids-containing cells were stained with perilipine staining while endothelial cells and pericytes were revealed by CD31 and aSMA stainings respectively. White squared image (a., b. and c.) are zoomed areas showing the organization of endothelial cells and aSMA+ cells inside spheroids. In both conditions, aSMA+ cells that aligned with CD31+ cells could be observed (white arrows). In C2 condition, peripheral aSMA+ cells can also be observed (white head arrows) independently to CD 31+ cells. Scale bar: 200 μm. All quantitative values are shown as mean +/−standard deviations. All statistical analyses were performed by two sample t-test. *p<0.05, **p<0.01, ***p<0.001, ****p<0.0001.

FIG. 3. Response of P0-SVF spheroids to canonical UCP1 inducers. Spheroids were differentiated for 21 days in C 2+SB4 medium and treated (Stim) or not (Ctrl) with UCP1 inducers for the last three days of culture. Gene expression analysis of brown adipocytes markers (UCP1, CIDEA, PGC1a). Fold changes are relatively expressed to Ctrl condition as mean +/−standard deviation (n=1).

### EXAMPLE

### Material & Methods

### Human Donors for Adipose Tissues Biopsies

Human Stromal Vascular Fraction (SVF) was isolated from abdominal dermolipectomy (plastic surgery department, CHU Toulouse, France) of female donors (body max index ranging from 22.3 to 27.9 kg/m²). The experimental protocols were approved by the French research ministry's institutional ethics committee (No: DC-2015-23-49) and informed consent was obtained from all subjects in accordance with institutional guidelines on human tissue handling and use.

### Isolation and Amplification of Stromal-Vascular Fraction Cells From Human Adipose Tissue

Adipose tissue was mechanically dissociated and enzymatically digested for 45 min at 37 °C, under stirring, using collagenase NB4 (Coger, Germany) at 13.6 U/mL in α-MEM (Life-Technologies, UK), supplemented with 0.1% (v/v) amphotericin B (Life-Technologies, UK), and 1% (v/v) streptomycin/penicillin (Life-Technologies, UK) hereafter named aMEM-ASP. After filtration on a 100 μm nylon net filter (Steriflip, Millipore, USA) and centrifugation (600 g, 10 min), cells were washed in aMEM-ASP and centrifuged again (600 g, 5 min). Cell pellet was resuspended in erythrocyte lysis buffer (eBioscience™ RBC Lysis Buffer Multi-species, Life-Technologies, UK) and incubated 5 min at RT to eliminate erythrocyte. Isolated Stromal Vascular Fraction cells (SVF-cells) were then centrifuged (600 g, 5 min) and resuspended in EGM2 (PromoCell, Germany) supplemented with ASP. Final cell solution was counted using a Malassez cell and seeded directly in suspension for spheroid formation or at 4000 cells/cm² in two-dimensions (2D) culture for further amplification. 2D cultures were maintained in EGM2, medium was changed every three days until they reached 80% confluency. The resulting amplified cells (P0-SVF) were used for spheroid formation or adipocyte differentiation in 2D cultures.

### Spheroid Formation

Spheroids were formed from either P0-SVF cells or from SVF cells. To promote cell aggregation, 50 000 cells were seeded in small volume (50 μL) of EGM2 medium in ultra-low attachment (ULA) 96-well round-bottom plates (Corning Incorporated Lifes Sciences, USA) and maintained overnight under stirring (150 rpm). For SVF cells, to further improve cell aggregation, cell seeding step was followed by plate centrifugation (600 g for 5 min). The following day, 150 μL of EGM2 was added in each well. Cells were maintained in proliferation medium until spheroid formation, i.e. five days for SVF-spheroids and one day for P0-SVF-spheroids.

### Adipocyte Cell Differentiation From 2D and 3D Cultures

Differentiation onset varied according to the type of cultures. For 2D culture, P0-SVF cells were first seeded at 80 000 cells/cm² on 0.1% gelatin (Sigma, USA) coated plates in EGM2 medium. Differentiation was then initiated when cells reached 100% confluency. For spheroids, differentiation was initiated once spheroids were formed. For both types of culture, cells were then differentiated for 21 days with appropriate adipogenic cocktails. Half of the medium was changed every three to four days. As a reference for white adipocyte differentiation, a standard adipogenic cocktail was used (Standard Cocktail). This standard cocktail consists of aMEM-ASP supplemented with 2% Foetal Bovine Serum (FBS, Life technologies, UK), 1 μM dexamethasone (Sigma, USA), 60 μM indomethacin (Sigma, USA), 2 μM rosiglitazone (Sigma, USA), 5 μg/mL insulin (Sigma, USA). 450 μM 3-isobutyl-1-methylxanthine (IBMX, Sigma, USA) was also added for the first three days of culture only. An adipogenic cocktail previously described by our team (Muller et al. 2019) to be compatible for both endothelial cells (ECs) maintenance and white adipocyte differentiation was also used. This first adipogenic Cocktail (C1), consists of α-MEM-ASP supplemented with 2% FBS, 5 μg/mL insulin, 10 μg/mL apotransferrin (Sigma, USA), 50 ng/ml bone morphogenetic protein 7 (BMP7, MiltenyiBiotec, France) with 0.2% intralipids (20% emulsion, Sigma, USA). A variation of adipogenic Cocktail 1 deprived from intralipids was also tested and is referred to as adipogenic Cocktail 2 (C2). When specified, the TGFβ pathway inhibitor SB431542 (MiltenyiBiotec, Germany), also referred to as SB4, was added to the adipogenic cocktail at a concentration of 5 μg/mL.

### RNA Extraction and Quantitative Relative Real Time PCR

An average of 24 spheroids or one well of a 12-wells plate for 2D monolayers were homogenized in QIAzol Lysis Reagent (Qiagen, USA). 3D culture samples were further disrupted for 2 min at 30 Hz using Tissue Lyser (Qiagen) to increase lysis efficiency. Total RNA was isolated using Phenol-chloroform extractions followed by Quick-RNA microprep kit procedure (Zymo Research, USA). Reverse transcription of 400 ng RNA into cDNA was performed using the high-capacity cDNA reverse transcription kit (Applied Biosystems, USA). cDNA was synthetized in a thermal cycler (2720 Applied Biosystems, USA) with the following program: 10 min at 25 °C, 120 min at 37 °C and 5 min at 85 °C cDNA was amplified by StepOne Real-Time PCR system for qPCR in 96 wells plate (Applied Biosystems, USA) using SYBR Green PCR Master Mix supplemented with 1/100e diluted cDNA and 375 nmol/L of primers. Ribosomal Protein Lateral Stalk Subunit P0 (RPLP0), Glucuronidase Beta (GUSB), Peptidylprolyl Isomerase A (PPIA) and Tyrosine 3-Monooxygenase/Tryptophan 5-Monooxygenase Activation Protein Zeta (YWAZ) were used as reference genes. Genes with cycles threshold (Ct)>35 were considered undetectable. Relative expression was calculated by the 2^{-ΔΔCT} method. The ΔCt was obtained by normalizing the mean expression values of each gene to the geometric mean of the reference genes. The ΔΔCt was calculated by normalizing conditions to 2D undifferentiated cells for 2D experiments or to undifferentiated spheroids for 3D culture experiments.

### Cell Viability Assay

### Spheroid Size Measurements

Imaging of spheroid size was performed during the culture process at indicated times using a Nikon eclipse TE2000-5 microscope with a 10× objective. Spheroid area was measured using Fiji software (National Institutes of Health, USA). Spheroids were measured for each time point per human sample. N human samples were analyzed.

### Iodure Propidium Staining

A 3D image-based cell viability quantification was conducted by staining free spheroids with 10 μg/mL propidium iodide (Invitrogen, USA) in culture medium for one hour at 37 °C. After three DPBS washes, samples were fixed with 4% paraformaldehyde. The fixed cultures were permeabilized and stained with DAPI. Samples were washed three times with D-PBS (30 min, RT) and cleared at least for 48 h with Scale S4 solution (Hama H. et al. Nat Neurosci. 2015) before imaging. All samples were imaged using a confocal microscope (LSM 880, Carl Zeiss, France). Optical slices were taken from the surface at 4.5 μm intervals, up to the depth of 200 um and presented as a vertical projection. For each optical slice, total number of nuclei and IP+ nuclei were quantified using imageJ. Prior to nuclei counting, DAPI signal was segmented using 2D Stardist pluging (Shmidt et al, 2018), a deep-learning-based method of 2D nucleus detection. The average viability percentage was calculated as the number of IP+ nuclei/DAPI+ nuclei per slice. Spheroids were measured for each condition per human sample. N human samples were analysed.

### DNA Quantification

DNA quantification was performed to assess cell proliferation and maintenance. DNA was extracted according to the blood and tissue DNA extraction kit (Qiagen) manual. Spheroids were washed with PBS 1× and lysed in 200 AL ALT/Proteinase K buffer overnight under mixing (800 rpm). To ensure the DNA purity, RNase A (Qiagen) was added to the samples and incubated for 2 min at RT before addition of 1:1AL/100 % ethanol mix. DNA was detected with the 1× Qubit™ High sensitivity dsDNA kit according to manufacturer's instructions. Fluorescent intensities were measured with Qubit 4.0 fluorometer (Invitrogen, CRCT, Toulouse). Data were expressed as ng of DNA/spheroid.

### Histology, Immunohistochemistry, and Immunofluorescence

2D and 3D cultures were fixed with 4% paraformaldehyde at RT. In the case of 3D culture, after D-PBS washing, samples were permeabilized and blocked in D-PBS solution supplemented with 1% Triton X-100 (Sigma, USA) and 3% horse serum (Jackson Immunoresearch, UK) for 3h at RT. Samples were then incubated with primary antibody in D-PBS solution supplemented with 1% horse serum and 1% Triton X-100, at the appropriate dilution, overnight at RT. After three washes in D-PBS, secondary antibodies coupled with Alexa-488, Alexa-594 or Alexa-647, (Life Technologies, UK), diluted at 1:500 in D-PBS supplemented with 1% horse serum and 1% Triton X-100 were added as specified, 3 h at RT. For lipid droplets staining, 2 μg/mL 493-Bodipy (Life Technologies, UK) was added to the solution. After D-PBS washes, nuclei were stained with 2 μg/mL DAPI, 1 h at RT (Sigma, USA). For 2D culture, the same protocol was used but incubation durations were shortened for the different steps. For 3D culture imaging, samples were cleared for at least 48 h in Scale S4 solution (Hama et al. 2015) composed of 40% (w/v) D-(-)-Sorbitol (Sigma, USA), 10% (w/v) glycerol (Euromedex, France), 4 M Urea (Sigma, USA), 0.2%Triton X-100, 20% (v/v) Dimethylsulfoxide (Sigma, USA). Samples were analyzed by confocal imaging (LSM 880, Carl Zeiss, France) and images were processed using Fiji software (National Institutes of Health, USA).

### Statistical Analysis

All results are presented as mean values of independent experiments, each from a different donor, ±standard deviation. The data were analyzed for normal distribution with kolmogorow-Smirnow test and homoscedasticity with Levene test. Significant differences among groups were evaluated with two samples t-test students or one-way analysis of variance (ANOVA-1) type III followed by post hoc analysis with Tukey's multiple comparison test. Significant fold differences compared to a reference control set to one were analyzed by one sample t-test. For qPCR analysis, statistical tests were performed on −ΔΔCt values as previously described. p-values <0.05 were considered significant.

### Treatment With UCP1 Inducers

Cells were treated three days prior to the end of the differentiation process with 100 nM rosiglitazone (Sigma, USA), 0.2 nM 3,3′,5-Triiodo-L-thyronine (T3, Sigma, USA), 0.1 μM all-trans retinoic acid protected from light (Sigma, USA), 200 μM 8-(4-Chlorophenylthio)-adenosine 3′,5′-cyclic monophosphate (8-CPT-cAMP, Abcam, UK). All-trans retinoic acid treatment was renewed every day until the end of the culture to overcome its molecular instability.

### Results

Development of a brown adipogenic medium preserving endothelial cells organization from human ASC in 2D culture. Vasculature is highly abundant in brown adipose tissue, even more than in white adipose depots (Xue, Y. et al., Cell Metabolism 18, 478-489, 2010). Therefore, we aimed to develop a brown/beige adipogenic medium promoting UCP1 expression and preserving endothelial cells development in a 3D context. To this end, we preliminary adjusted these conditions in 2D cultures. After amplification in EGM2 medium that allows both to expand adipose and endothelial progenitors (Min SY et al., Nat Med., 2016), P0-SVF cells were submitted to different adipogenic cocktails for 21 days (FIG. 1). We tested an adipogenic cocktail, referred to as cocktail 1 (C1), previously developed by our team to promote white adipocyte differentiation from human ASC while preventing the loss of endothelial cells (Muller S et al., Sci Rep. 2019). In this previous study, intralipids were used as a physiological-like adipogenic inducer to generate hypertrophic adipocytes. Thus, we also tested a variation of C1 medium deprived of free fatty acid, described in the following sections as adipogenic cocktail 2 (C2) to favor browning differentiation. Standard cocktail was used as a reference for induction of white adipocyte differentiation.

C2 medium was the most efficient to preserve endothelial cells as demonstrated by the increase of Cd31 endothelial cell expression (FIG. 1A) and CD31 staining (FIG. 1B). Although to a lesser degree compared to standard and C1 conditions, C2 medium was sufficient to induce an increase in adiponectin (Adipoq) expression, a late marker of adipogenesis (FIG. 1A) and lipid accumulation revealed by BODIPY staining (FIG. 1B). Interestingly, the maintenance of endothelial cells with C2 medium correlated with significantly higher Ucp1 expression of differentiated ASCs (FIG. 1A) suggesting C2 cocktail as the best medium to induce brown/beige adipocyte differentiation.

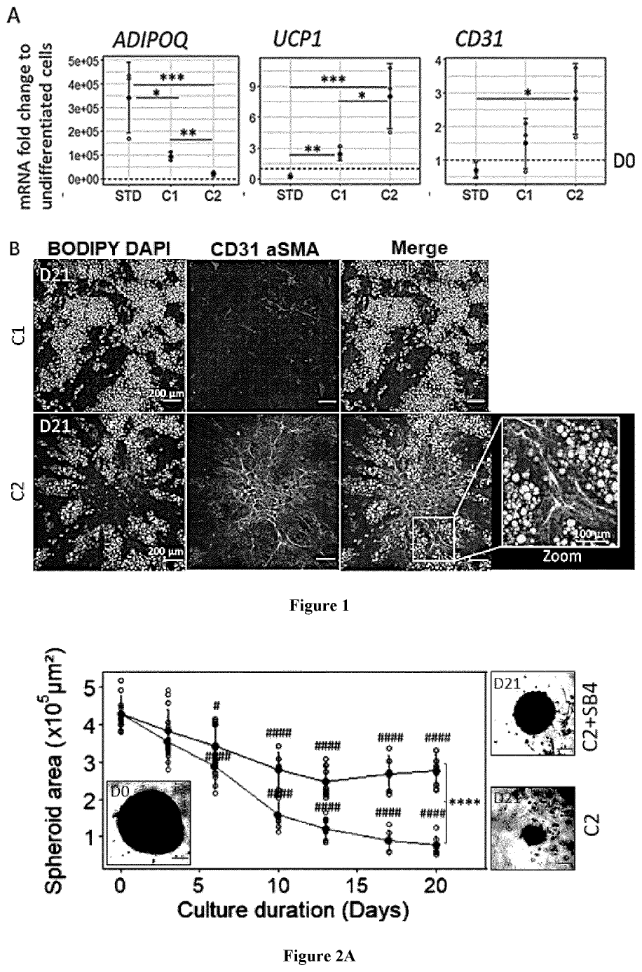

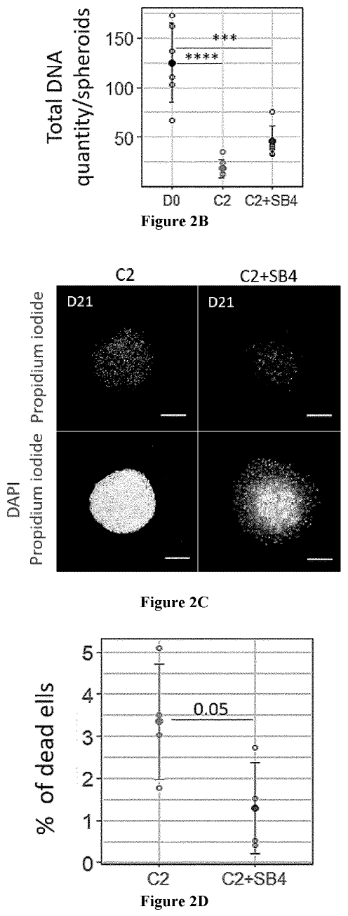

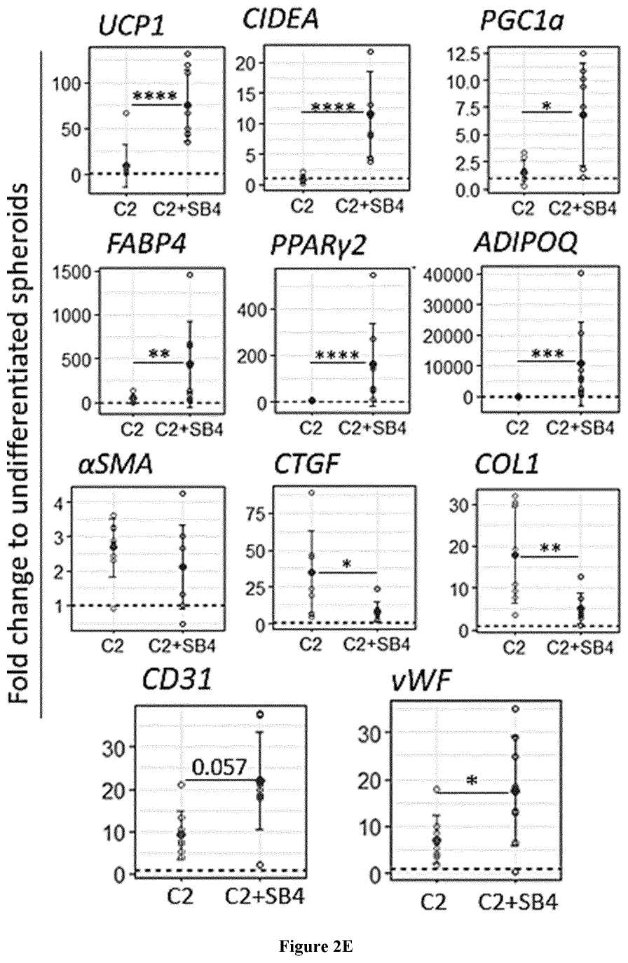

Development of a biochemical environment to induce brown adipogenesis of ASC from human adipose tissue while preserving endothelial cells organization in 3D spheroids culture. We then assessed if C2 medium could promote brown/beige adipocyte differentiation and vascular formation of P0-SVF cells cultivated as spheroids. After 2D cell expansion in EGM2, P0-SVF cells were harvested and seeded in ultra-low adherence plates to produce spheroids. Once formed, P0-SVF spheroids were cultivated for 21 days in the selected adipogenic medium. An important decrease of spheroid size (FIG. 2A) associated with a decrease in DNA content (FIG. 2B) was observed from day 0 to day 21 of differentiation suggesting cell loss. This decrease in size was associated with a decrease in DNA content (FIG. 2C-D) suggesting cell loss although propidium iodide staining revealed 3% of dead cells at day 21. Contrary to what was observed in 2D culture, cultivating the spheroid within the adipogenic medium was not sufficient to induce adipocyte differentiation of P0-SVF spheroids as revealed by an absence of adipocyte marker expressions (FIG. 2E, C2 condition) and lipid-containing cells (FIG. 2F, C2 condition). Regarding angiogenic potential of P0-SVF spheroids, we observed that C2 medium supported self-organization of CD31+ endothelial cells into pseudo-vascular networks aligned with perivascular smooth muscle cells expressing SMA marker (FIG. 2F, C2 condition and panel b). The presence of peripheral aSMA+ cells not aligned with CD31+ cells (FIG. 2F, C2 condition and panel a) associated with the increase of expression of collagen 1 (COL1a) and connective tissue growth factor (CTGF), compared to undifferentiated spheroid, seemed to indicate rather the induction of myofibroblast differentiation (FIG. 2E-F). Because the TGF-β pathway favors ASC differentiation into myofibroblasts at the expense of adipogenesis (Chignon-Sicard B., Sci Rep. 2017), we treated spheroids with an inhibitor of the TGF-β receptor (SB431542) all along the differentiation process.

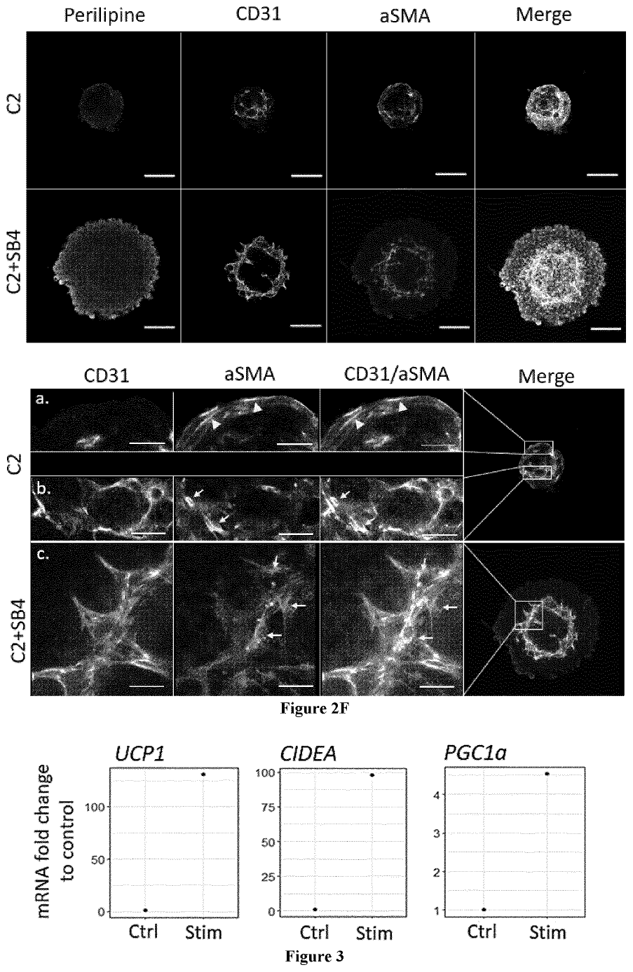

We observed that TGF-β inhibition led to better maintenance of spheroids with differentiation regarding spheroid size (FIG. 2A), DNA content (FIG. 2B) and cell death (FIG. 2C-D). Treatment with SB431542 increased gene expression of early and late adipogenesis markers including Peroxisome proliferator-activated receptor gamma isoform-2 (PPARg2), fatty acid binding protein 4 (FABP4) and adiponectin (ADIPOQ) (FIG. 2E). Expression of UCP1 and additional brown/beige adipocytes markers including cell death-inducing DFFA-like effector A (CIDEA) and Peroxisome proliferator-activated receptor-y coactivator (PGC1a) was also increased in the presence of SB431542. Interestingly, this was associated with a decreased expression of myofibroblasts markers such as CTGF and COL1 a. Immunofluorescence experiments showed that TGF-β inhibition decreased the presence of aSMA+ cells at the periphery of the spheroids while promoting the appearance of adipocytes (FIG. 2F). Interestingly TGF-β inhibition did not seem to interfere with the development of perivascular aSMA+ cells that aligned with CD31+ endothelial cells inside the spheroid. Results depicting a functional assessment of P0-SVF spheroids response to UCP1 inducers are shown in FIG. 3.

### CONCLUSION

The addition of TGF-β inhibitor in the C2 cocktail significantly increases pseudo-vascular formation and adipocyte brown/beige differentiation in 3D cell aggregates. Among the many advantages conferred by these new methods, it should be noted that the culture of the 3D cell aggregates can be done without any scaffold.

### REFERENCES

Throughout this application, various references describe the state of the art to which this invention pertains. The disclosures of these references are hereby incorporated by reference into the present disclosure.

1. An in vitro method for generating a prevascularized 3D cell aggregate comprising the step of contacting mesenchymal stroma/stem cells and endothelial cells with a differentiation medium and a TGF-β inhibitor.

2. The in vitro method according to claim 1 wherein the mesenchymal stroma/stem cells are adipose tissue-derived stem cells.

3. The in vitro method according to claim 2 wherein the adipose tissue-derived stem cells are white adipose tissue-derived stem cells.

4. The in vitro method according to claim 1 wherein the mesenchymal stroma/stem cells and endothelial cells are derived from a Stromal Vascular Fraction (SVF).

5. The in vitro method according to claim 1 wherein the differentiation medium comprises at least one adipogenic agent and/or at least one browning agent.

6. The in vitro method according to claim 1 wherein differentiation medium comprises α-MEM; a mix of growth factor, insulin, and apotransferrin; and a bone morphogenic protein.

7. An in vitro method for generating a prevascularized 3D cell aggregate comprising the steps of:
contacting a Stromal Vascular Fraction (SVF) comprising mesenchymal stroma/stem cells and endothelial cells with an amplification medium to generate an amplified population of cells;
seeding said amplified population of cells in an ultra-low adherence surface; and
contacting said seeded amplified population of cells with a differentiation medium according to claim 5, and a TGF-β inhibitor.

8. The in vitro method according to claim 1, wherein the TGF-β inhibitor is SB431542.

9. The in vitro method according to claim 1 further comprising the steps of:
contacting a SVF comprising mesenchymal stroma/stem cells and endothelial cells with an amplification medium to generate an amplified population of cells;
seeding said amplified population of cells in an ultra-low adherence surface; and
contacting said seeded amplified population of cells with a differentiation medium comprising α-MEM-ASP, foetal bovine serum, insulin, apotransferin, BMP7 and SB431542,
whereby the 3D prevascularized cell aggregate thus generated is a prevascularized brown/beige adipocytes-containing 3D cell aggregate.

10. A prevascularized 3D cell aggregate obtained with the in vitro method according to claim 1.

11. A method of treating a metabolic disorder in a subject in need thereof, comprising administering to the subject a therapeutically effective dose of at least one prevascularized 3D cell aggregate of claim 10.

12. A method of conducting tissue transplantation in a subject in need thereof, comprising transplanting at least one prevascularized 3D cell aggregate of claim 10.

13. The method according to claim 11, wherein the prevascularized 3D cell aggregate is a prevascularized brown/beige adipocytes-containing 3D cell aggregate.

14. An in vitro method of screening a compound that modulates brown/beige adipose tissue activity comprising:
contacting the prevascularized brown/beige adipocytes-containing 3D cell aggregate obtained by the method of claim 9 with a compound suspected of modulating adipose tissue activity, and
monitoring an effect of said compound on activity of said prevascularized brown/beige adipocytes-containing 3D cell aggregate.

15. The in vitro method of claim 14 wherein the monitoring step comprises monitoring UCP1 expression.

16. A medium for producing a prevascularized brown/beige adipocytes-containing 3D cell aggregate comprising α-MEM-ASP, foetal bovine serum, insulin, apotransferrin, BMP7 and SB431542.

17. (canceled)

18. An in vitro method of increasing viability of a 3D cell aggregate, comprising contacting the 3D cell aggregate with a TGF-β inhibitor.

19. The in vitro method according to claim 18 wherein the 3D cell aggregate is a spheroid.

20. The in vitro method according to claim 18 wherein the 3D cell aggregate comprises mesenchymal stroma/stem cells and endothelial cells.

21. The in vitro method according to claim 18, wherein the 3D cell aggregate is a prevascularized 3D cell aggregate comprising mesenchymal stroma/stem cells.
